(19) United States
(12) Patent Application Publication
(10) Pub. No.: US 2026/0060601 A1
(43) Pub. Date: Mar. 5, 2026

(54) SYSTEM AND METHOD FOR ANALYZING COGNITIVE FUNCTION

(71) Applicant: Xiao-ke Gao, Riverside, CT (US)

(72) Inventor: Xiao-ke Gao, Riverside, CT (US)

(21) Appl. No.: 19/097,204

(22) Filed: Apr. 1, 2025

Related U.S. Application Data
(60) Provisional application No. 63/688,509, filed on Aug. 29, 2024.

(51) Int. Cl. A61B 5/00; A61B 5/11

(52) U.S. Cl. CPC A61B 5/4088; A61B 5/0077; A61B 5/112

(57) ABSTRACT

A system and method are provided for diagnosing mild cognitive impairment. The system and method utilize one or more computing devices to instruct a user to record the performance of one or more neuromuscular commands, such as finger tapping, heal tapping, or gait movements. The system and method analyze one or more audio components of the performance to determine one or more attributes, such as a speed of performance, a rhythm of performance, a variability in performance, or a volume intensity of performance. The system and method utilize the one or more attributes to generate at least one score which can be utilized to determine if cognitive impairment exists in the user, or to monitor progression of cognitive impairment in the user.

### CROSS-REFERENCE TO RELATED APPLICATIONS

This application claims the benefit of priority of U.S. provisional application number 63/688,509, filed Aug. 29, 2024, the contents of which are herein incorporated by reference.

### BACKGROUND OF THE INVENTION

### Field of Endeavor

The present invention relates to diagnostic systems, and more particularly, to a system for analyzing human movement utilizing audio files for assessing cognitive function of a user.

### Background of Related Art

Evaluation of a patient's cognitive status is a critical element of primary care as cognitive impairments can otherwise go unnoticed and undiagnosed for years. One mechanism for testing cognitive status of a patient includes placing sensors on the patient and utilizing the sensors to analyze movements to determine levels of cognitive impairment. However, this mechanism is disadvantageous because it requires the patient to go to an in-office setting where sensors are available, which is time-consuming, costly, and inconvenient, all of which may prevent a patient from seeking diagnosis and treatment.

As can be seen, there is a need for improve cognitive testing system that does not require sensors, which can be performed remotely to remove barriers to diagnosis.

### SUMMARY OF THE INVENTION

In one aspect of the present invention a system is provided for diagnosing mild cognitive impairment. In aspects of the present invention the system includes at least one processor, and at least one memory, the at least one memory storing instructions that when executed perform a method for diagnosing mild cognitive impairment. The system of the present invention can provide at least one instruction to a user to perform one or more neuromuscular tasks, such as finger tapping, heal tapping, and/or gait movements. The system can receive at least one file having data thereon indicative of performance of the at least one instruction by the user. The system of the present invention can analyze the at least one file to determine one or more attributes associated with the performance of the one or more neuromuscular tasks, and can generate at least one score indicative of the quality of performance of the one or more neuromuscular tasks. The at least one score can be output by the system as a means for diagnosing mild cognitive impairment and/or tracking a progress of one or more mild cognitive impairments.

In another aspect of the present invention a method for diagnosing mild cognitive impairment is provided. In aspects of the present invention the method can be performed on a computing device, such as a smart phone with one or more sensors. The method of the present invention can provide at least one instruction to a user to perform one or more neuromuscular tasks, such as finger tapping, heal tapping, and/or gait movements. The method can receive at least one file having data thereon indicative of performance of the at least one instruction by the user. The method of the present invention can analyze the at least one file to determine one or more attributes associated with the performance of the one or more neuromuscular tasks, and can generate at least one score indicative of the quality of performance of the one or more neuromuscular tasks. The at least one score can be output by the system as a means for diagnosing mild cognitive impairment and/or tracking a progress of one or more mild cognitive impairments.

### BRIEF DESCRIPTION OF THE DRAWINGS

FIG. 1 is a block diagram of an embodiment of a system for analyzing cognitive function, according to aspects of the present disclosure; and

FIG. 2 is a flow diagram of an embodiment of a method for analyzing cognitive function of a user, according to aspects of the present invention.

### DETAILED DESCRIPTION OF THE DISCLOSURE

The following detailed description is of the best currently contemplated modes of carrying out exemplary embodiments of the disclosure. The description is not to be taken in a limiting sense but is made merely for the purpose of illustrating the general principles of the disclosure, since the scope of the disclosure is best defined by the appended claims.

Typical systems or methods for analyzing cognitive function of a user require the user to visit an in-office setting, be hooked up to sensors, and perform a plurality of tasks as directed by a medical professional. However, these systems provide barriers to diagnosis because they are time consuming, costly, and inconvenient.

Broadly, an embodiment of the present disclosure provides a system for analyzing cognitive function of a user utilizing audio files. The present invention can include a computing device, such as a smartphone, which can record audio of the user performing a series of movement patterns, as directed. In the present invention the recorded audio can be transmitted to a computing system for analysis. In the present invention the audio file can be analyzed to provide a score associated with a user's cognitive function, which can be reported to a medical professional. Advantageously, the present invention is non-invasive and can be performed anywhere a computing device with audio recording capabilities is present, thereby lowering barriers and disadvantages associated with prior art cognitive testing systems and methods.

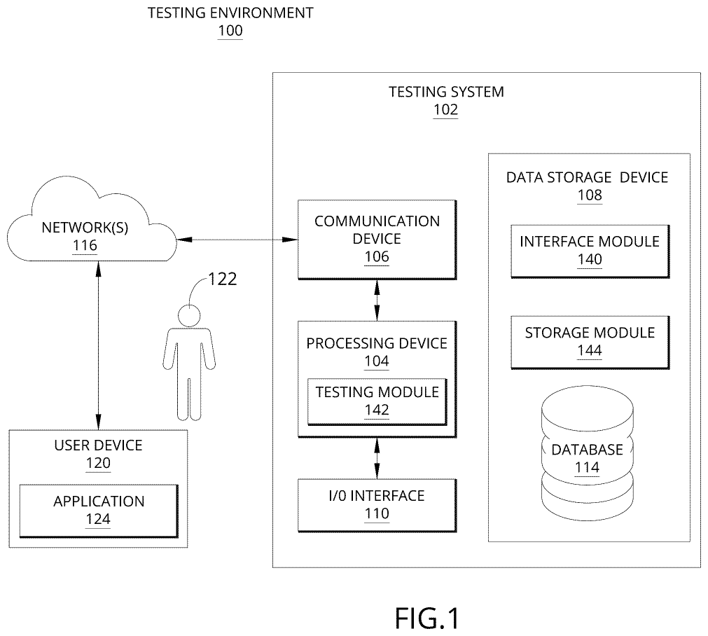

Referring now to FIG. 1, FIG. 1 illustrates an embodiment of a Testing Environment 100 for analyzing cognitive function, according to aspects of the present disclosure. While FIG. 1 illustrates various components of environment 100, additional components can be added, and existing components can be removed.

As illustrated in FIG. 1, Testing System 102 includes one or more processing devices, herein processing device 104, coupled to a communication device 106. The processing device 104 is also coupled to a memory device 108, and an input/output (“I/O”) interface 110. In embodiments, the communication device 106 enables the Testing System 102 to communicate with other devices and systems via one or more networks 116. The Testing System 102 can communicate with a user device 120 via the network 116. A user 122 can utilize the user device 120 to communicate with the Testing System 102. The user device 120 can include one or more electronic devices such as a laptop computer, a desktop computer, a tablet computer, a smartphone, a thin client, a smart appliance, and the like. While FIG. 1 illustrates one user device 120, Testing environment 100 can include multiple user devices operated by the user 122 or operated by other users.

According to the aspects of the present disclosure, the Testing System 102 enables the user 122, operating a copy of an application 124 executing on the user device 120, to communicate with the Testing System 102 and leverage the service provided by the Testing System 102. The Testing System 102 is configured to analyze audio files of user 122 movement to determine cognitive status of user 122. While Testing System 102 and user device 120 with application 124 are illustrated as separate components, it is envisioned that Testing System 102 can be integrated as a singular application, i.e. application 124, and with the architecture implemented as user device 120.

To perform the process described herein, the Testing System 102 can store and execute an Interface module 140, a Testing module 142, and a Storage module 144 to perform the processes and methods described herein. The Interface module 140, the Testing module 142, and the Storage module 144 can be stored in the memory device 108. The Interface module 140, the Testing module 142, and the Storage module 144 can include the necessary logic, instructions, and/or programming to perform the processes and methods described in further detail below. The Interface module 140, the Testing module 142, and the Storage module 144 can be written in any programming language.

In embodiments, the application 124 can be a specifically designed application that operates with the Testing System 102 to perform the processes and methods described herein. In embodiments, the application 124 can be a third-party application, such as a web browser, that communicates with the Testing System 102 to perform the processes and methods described herein. Additionally, the application 124 can include the necessary logic, instructions, and/or programming of Testing System 102 to perform the processes and methods described in further detail below. The memory device 108 can also include one or more databases 114 that store information and data associated with the process and methods described below in further detail.

According to aspects of the present disclosure, the Testing System 102, for example, via the Interface module 140, provides unique interfaces that allow the user 122 receive instructions on a movement pattern to be performed, receive a movement pattern, such as typing on a screen, tapping on a screen, record audio files of a movement pattern, transmit recorded audio files, etc. The Interface module 140 operates to generate and provide graphical user interfaces (GUIs) to the application 124, for example, menus, widgets, text, images, fields, etc., as described below in further detail. The GUIs generated by the Interface module 140 can be interactive. The Testing System 102, for example, via the Interface module 140, also provide one or more application programming interface (APIs) that provide connection points for one or more application, e.g., the application 124.

In embodiments, the Interface module 140 can implement voice control aspects into the interfaces provided. For example, the user can navigate the interfaces of the Testing System 102 using the audio input device of the user device 120. The interface module 140 can implement one or more chat-bots to deliver conversational input and output to a user.

According to aspects of the present disclosure, the Testing System 102, for example, via the Testing module 140, provides an algorithm by which recorded audio files of user 122 performing a movement pattern can be analyzed. In embodiments, Testing module 140 through the algorithm can analyze movements of a user utilizing audio processing, to determine a score associated with a recorded audio file. In embodiments, the score can indicated a cognitive status associated with user 122.

The processing device 104, the communication device 106, the memory device 108, and the I/O interface 110 can be interconnected via a system bus. The system bus can be and/or include a control bus, a data bus, and address bus, and so forth. The processing device 104 can be and/or include a processor, a microprocessor, a computer processing unit (“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and so forth. As used herein, “processor,” “processing component,” “processing device,” and/or “processing unit” can be used generically to refer to any or all of the aforementioned specific devices, elements, and/or features of the processing device. While FIG. 1 illustrates a single processing device 104, the Testing System 102 can include multiple processing devices 104, whether the same type or different types.

The memory device 108 can be and/or include computerized storage medium capable of storing electronic data temporarily, semi-permanently, or permanently. The memory device 108 can be or include a computer processing unit register, a cache memory, a magnetic disk, an optical disk, a solid-state drive, and so forth. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device. While FIG. 1 illustrates a single memory device 108, the Testing system 102 can include multiple memory devices 108, whether the same type or different types.

The communication device 106 enables the Testing System 102 to communicate with other devices and systems. The communication device 106 can include, for example, a networking chip, one or more antennas, and/or one or more communication ports. The communication device 106 can generate radio frequency (RF) signals and transmit the RF signals via one or more of the antennas. The communication device 104 can generate electronic signals and transmit the RF signals via one or more of the communication ports. The communication device 106 can receive the RF signals from one or more of the communication ports. The electronic signals can be transmitted to and/or from a communication hardline by the communication ports. The communication device 106 can generate optical signals and transmit the optical signals to one or more of the communication ports. The communication device 106 can receive the optical signals and/or can generate one or more digital signals based on the optical signals. The optical signals can be transmitted to and/or received from a communication hardline by the communication port, and/or the optical signals can be transmitted and/or received across open space by the communication device 106.

The communication device 106 can include hardware and/or software for generating and communicating signals over a direct and/or indirect network communication link. As used herein, a direct link can include a link between two devices where information is communicated from one device to the other without passing through an intermediary. For example, the direct link can include a Bluetooth™ connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

The Testing System 102 can communicate with one or more network resources via the network 116. The one or more network resources can include external databases, social media platforms, search engines, file servers, web servers, or any type of computerized resource that can communicate with the Testing System 102 via the network 116.

As described above, the Testing System 102 can include hardware components to perform the processes described herein. In embodiments, one or more of components, hardware, and/or functionality of the Testing System 102 can be hosted and/or instantiated on a “cloud” or “cloud service. ” As used herein, a “cloud” or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

In embodiments, the components and functionality of the Testing System 102 can be and/or include a “server” device. The term server can refer to functionality of a device and/or an application operating on a device. The server device can include a physical server, a virtual server, and/or cloud server. For example, the server device can include one or more bare-metal servers such as single-tenant servers or multiple-tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers.

Various aspects of the systems described herein can be referred to as “information,” “content,” and/or “data. ” Content and/or data can be used to refer generically to modes of storing and/or conveying information. Accordingly, data can refer to textual entries in a table of a database. Content and/or data can refer to alphanumeric characters stored in a database. Content and/or data can refer to machine-readable code. Content and/or data can refer to images. Content and/or data can refer to audio and/or video. Content and/or data can refer to, more broadly, a sequence of one or more symbols. The symbols can be binary. Content and/or data can refer to a machine state that is computer-readable. Content and/or data can refer to human-readable text.

Various of the devices in the Testing Environment 100, including the Testing System 102 and/or the user device 120 can provide I/O devices for outputting information in a format perceptible by a user and receiving input from the user. For example, the Testing System 102 can communicate with the I/O devices via the I/O interface 110. The I/O devices can display graphical user interfaces (“GUIs”) generated by the Testing System 102. The I/O devices can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The I/O devices can include an acoustic element such as a speaker, a microphone, and so forth. The I/O devices can include a button, a switch, a keyboard, a touch-sensitive surface, a touchscreen, a camera, a fingerprint scanner, and so forth. The touchscreen can include a resistive touchscreen, a capacitive touchscreen, and so forth.

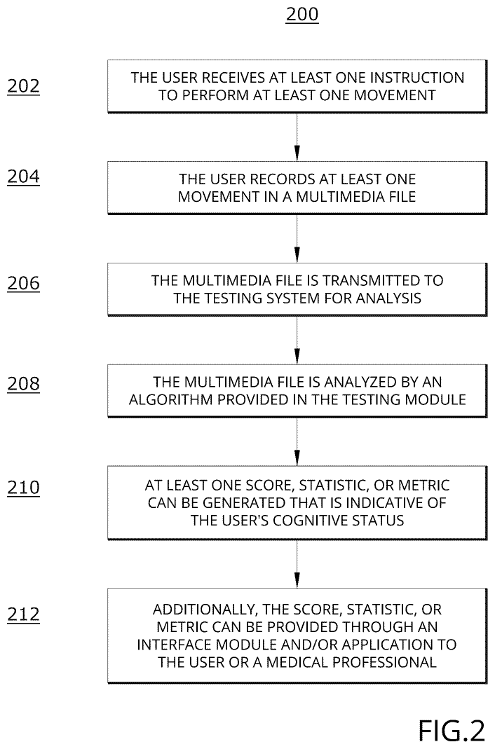

FIG. 2 illustrates method 200 for analyzing cognitive status of a user, according to aspects of the present disclosure. While FIG. 2 illustrates various stages of the method 200, additional stages can be added, and existing stages can be removed and/or reordered.

Method 200 can begin at step 202 where a user 122 can receive at least one instruction to perform at least one movement. In embodiments, Testing System 102 through Testing module 142 and Interface module 140 can provide the at least one instruction. In embodiments, the at least one instruction can include instructions for finger-tapping, or foot-movement, and/or gait movements but is not so limited. In an exemplary embodiment, user device 120 running application 124 can provide the at least one instruction through one or more user interface elements. For example, application 124 can instruct user 122 to tap one or more of their fingers on a surface, or alternatively tap one or more of their feet on a surface. In embodiments, the one or more instructions can be provided as a means for diagnostic testing of mild cognitive impairment, or alternatively, as a part of an exercise regimen configured to prevent, or slow, mild cognitive impairment.

At step 204, user 122 can perform the one or more instructions which can be recorded for further analysis. In embodiments, user device 120 can record the performance of the one or more instructions using one or more I/O devices, such as a microphone, camera, or other audio recording I/O device. For example, application 124 can record the performance of the one or more instructions using the one or more I/O devices of user device 120 and can save the record as one or more files, such as one or more multimedia files. In embodiments, the one or more multimedia files can be audio, video, etc., but is not so limited. In an exemplary embodiment, user device 120 can record the performance of the one or more instructions, by user 122, utilizing one or more audio sensors, as the one or more I/O devices, and can save the record as one or more audio files, in known audio file formats.

At step 206, the one or more files can transmitted to Testing system 102 for analysis. In embodiments, transmission can occur via e-mail, SMS message, MMS message, or through transmission functionality provided by application 124, but is not so limited. In alternative embodiments, Testing system 102 be resident on user device 120, with functionality incorporated into application 124, and in such cases transmission of the one or more files can occur via a system bus, as known in the art.

At step 208, the one or more files can be analyzed by Testing module 142. In embodiments, Testing module 142 can analyze the one or more files for one or more attributes such as, but not limited to, speed, rhythm, and/or volume intensity of the one or more performed instructions. In embodiments, one or more signal processing algorithms can be provided to pre-process one or more data items contained in the one or more files, such as filtering, normalizing, etc. In an exemplary embodiment, the one or more signal processing algorithms can include one or more noise filtering algorithms configured to remove noise from the one or more files.

Once pre-processed, the one or more files can be processed by one or more additional signal processing algorithms to determine the one or more attributes. In embodiments, the one or more additional algorithms can include audio signal processing algorithms for estimating acoustic metrics, labeling and augmenting audio data sets, and extracting audio features. Specifically, the one or more additional signal processing algorithms can be utilized to determine one or more taps, of an extremity, from the one or more files, which can be utilized in determining the one or more attributes. In embodiments, the one or more taps can be determined by comparing one or more amplitudes and comparing those amplitudes to one or more thresholds. In an exemplary embodiment, the one or more files contain audio signals which can be processed by the one or more additional signal processing algorithms to determine one or more taps as portions of the audio signals above an amplitude threshold.

In embodiments, once one or more taps have been detected, Testing module 142 can utilize the one or more taps to determine the one or more attributes. In embodiments, Testing module 142 can determine as the one or more attributes at least one of: a speed between the one or more taps (i.e. speed), a standard deviation for the speed between the one or more taps (i.e. rhythm), an amplitude mean of the one or more taps (i.e. volume intensity), and an amplitude mean standard deviation.

In embodiments, Testing module 142 can utilize the one or more attributes to determine a cognitive status of the user 122 by analyzing one or more of a performance consistency and variance to reflect cognitive status of the user 122.

In exemplary embodiments, the one or more attributes can be one or more a rhythm of the user 122, a speed of action of the user 122, and/or a volume intensity of an action of the user 122. In embodiments, the one or more attributes can be compared by Testing module 142 to prior performances by user 122, or alternatively, to thresholds, to determine if user 122 has mild cognitive impairment. In embodiments, mild cognitive impairment can be determined by changes in the one or more attributes, such as slowing of speed, irregularity in performance, and/or diminished volume intensity.

For example, Testing Module 142 can utilize the one or more attributes to determine a standard deviation of inter-action rhythm, such as inter-finger tapping rhythm. In embodiments, speed of the one or more performed instruction in combination with the standard deviation of inter-action rhythm can indicate how well the one or more instructions was performed, which can be used by Testing module 142 to determine the presence, or absence, of mild cognitive impairment. In embodiments, inter-action rhythm can be an indication of brain-controlled attention and consistency of the user 122. Furthermore, volume intensity can be indicative fine motor function/movement, and/or coordination. In embodiments, if the one or more instructions is a heal tap or gait instruction, the one or more attributes can be indicative of neuromuscular coordination and/or balance.

At step 210, at least one score, statistic, or metric can be generated which is indicative of user 122 cognitive status. In embodiments, the at least one score, statistic, or metric, can be calculated based on the analysis of the one or more attributes, as described above. In embodiments, one or more thresholds can be provided to determine the at least one score, statistic, or metric. For example, speed between taps (in milliseconds) and a corresponding standard deviation of speed between taps (in milliseconds) can be used to determine the at least one score, statistic, or metric. In the exemplary scenario, a speed between taps of less than 500 milliseconds with a standard deviation of less than 10 milliseconds can indicate excellent performance, a speed between taps of less than 550 milliseconds with a standard deviation of above 10 milliseconds but less than 25 milliseconds can indicate good performance, a speed between taps of less than 550 milliseconds with a standard deviation of above 25 milliseconds but less than 40 milliseconds can indicate moderate performance, a speed between taps of greater than 550 milliseconds with a standard deviation of above 40 milliseconds but less than 50 milliseconds can indicate poor performance, and a speed between taps of greater than 600 milliseconds with a standard deviation of above 50 can indicate poor performance. Additionally, the at least one score, statistic, or metric can be provided through interface module 140 and/or application 124 to user 122 or a medical professional. In embodiments, the at least one score, statistic, or metric, can be utilized to diagnose mild cognitive impairment, such as dementia, and/or monitor the progress of one or more cognitive impairment conditions.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

What is claimed is:

1. A system for diagnosing mild cognitive impairment, comprising:
at least one processor, and at least one memory, the at least one memory storing instructions that when executed perform a method, the method comprising:
providing, to a user, at least one instruction;
receiving, using one or more sensors of the system, at least one file having at least one data indicative of performance of the at least one instruction by the user;
analyzing, by the at least one processor, the at least one file to determine one or more attributes;
generating, based on the analysis, at least one score using the one or more attributes; and
outputting, by a user interface, the at least one score.

2. The system of claim 1, wherein the at least one instruction includes at least one of:
an instruction to perform at least one finger tap;
an instruction to perform at least one heal tap; or
an instruction to perform at least one gait movement.

3. The system of claim 1, wherein the one or more sensors includes: a microphone, and/or a video camera.

4. The system of claim 1, wherein the at least one file is a multi-media file.

5. The system of claim 1, wherein the analyzing the at least one file to determine one or
more attributes, further comprises:
performing signal analysis on the at least one file to determine the one or more attributes, wherein the one or more attributes is at least one of: a speed, a rhythm, a variability, and/or a volume intensity associated with the at least one instruction.

6. A method of diagnosing mild cognitive impairment, comprising:
providing, to a user, at least one instruction;
receiving at least file one having at least one data indicative of performance of the at least one instruction by the user;
analyzing the at least one file to determine one or more attributes;
generating, based on the analysis, at least one score using the one or more attributes; and
outputting the at least one score.

7. The method of claim 5, wherein the at least one instruction includes at least one of:
an instruction to perform at least one finger tap;
an instruction to perform at least one heal tap; or
an instruction to perform at least one gait movement.

8. The method of claim 5, wherein the at least one file is a multi-media file.

9. The method of claim 5, wherein the analyzing the at least one file to determine one or more attributes, further comprises:
performing signal analysis on the at least one file to determine the one or more attributes, wherein the one or more attributes is at least one of: a speed, a rhythm, a variability, and/or a volume intensity associated with the at least one instruction.
